(19) United States
(12) Patent Application Publication
(10) Pub. No.: US 2024/0408265 A1
(43) Pub. Date: Dec. 12, 2024

(54) ULTRAVIOLET LIGHT STERILIZATION SYSTEMS

(71) Applicant: William Clayton Johnson, Auburndale, FL (US)

(72) Inventor: William Clayton Johnson, Auburndale, FL (US)

(21) Appl. No.: 18/331,168

(22) Filed: Jun. 7, 2023

Related U.S. Application Data
(60) Provisional application No. 63/328,525, filed on Apr. 7, 2022.

(51) Int. Cl. A61L 9/20; B60H 1/00

(52) U.S. Cl. CPC A61L 9/20; B60H 1/00364; A61L 2209/12; A61L 2209/16

(57) ABSTRACT

UV sterilization systems for vehicle air conditioning systems, water filtration, and commercial dishwasher and cutting board sterilization are described.

### RELATED APPLICATIONS

This application claims the benefit of U.S. Application No. 63/328,585, entitled “Ultraviolet Light Sterilization Systems,” and filed on Apr. 7, 2022, which is incorporated herein by reference.

### FIELD

Some implementations relate generally to ultraviolet (UV) light sterilization system, and, more particularly, to UV sterilization systems for vehicle air conditioning systems, water filtration, and commercial dishwasher and cutting board sterilization.

### BACKGROUND

Conventional recreational vehicle (or marine or other vehicle) air conditioning systems may circulate unsterilized air within the vehicle.

Embodiments were conceived in light of the above-mentioned problems and limitations, among other things. The background description provided herein is for the purpose of generally presenting the context of the disclosure. Work of the presently named inventor(s), to the extent it is described in this background section, as well as aspects of the description that may not otherwise qualify as prior art at the time of filing, are neither expressly nor impliedly admitted as prior art against the present disclosure.

### BRIEF DESCRIPTION OF THE DRAWINGS

FIG. 1 is a diagram of an example recreational vehicle air conditioning system.

FIG. 2 is a diagram showing an example recreational vehicle air conditioning system having a UV sterilization component in accordance with some implementations.

FIG. 3 is a diagram showing an example UV sterilization component in accordance with some implementations.

### DETAILED DESCRIPTION

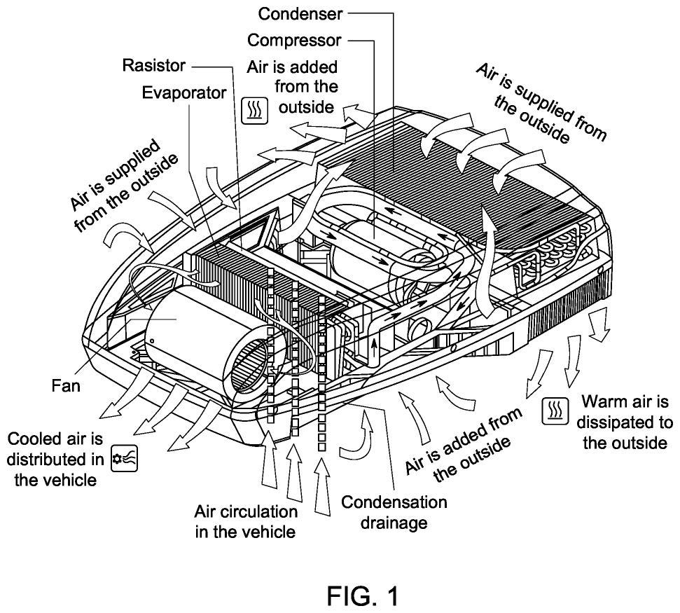

As shown in FIG. 1, a compressor compresses coolant and circulates the compressed coolant through a condenser coil while outside air is circulated over the condenser coil to remove heat from the condenser coil and dissipate the heat to the outside. Also, air can be added from the outside to the air being circulated within the vehicle.

The air in the vehicle or being brought in from the outside may have germs such as bacteria or viruses, or other pollutants such as mold spores.

Air in the vehicle is circulated over an evaporator coil to absorb heat from the interior air from the vehicle during the process of the coolant evaporating.

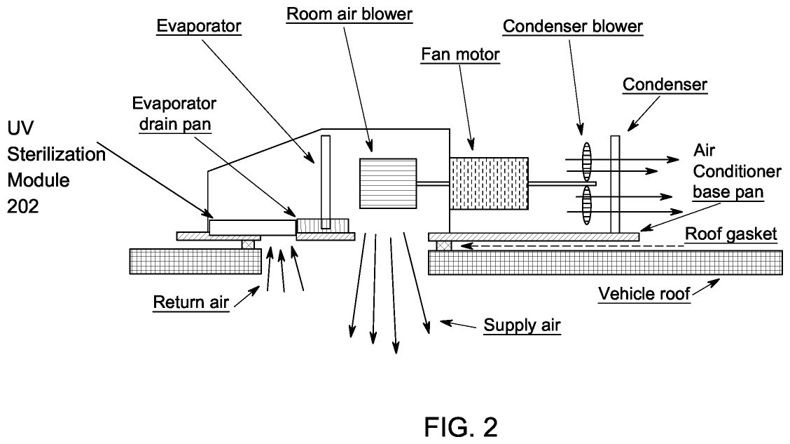

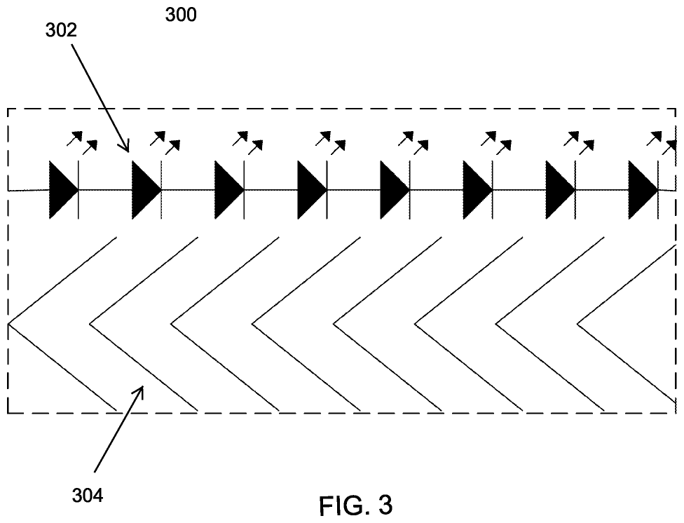

FIG. 2 is a diagram showing a recreational vehicle air conditioning system having a UV sterilization module in accordance with some implementations. The UV sterilization module 202 can include a UV tube in the vortex of the vehicle air conditioning unit. Further, as shown in FIG. 3, the UV sterilization module 202 can be constructed such that the incoming air is exposed to UV light at a level that helps inhibit or destroy microorganisms in the air, and in a manner such that the UV light is not visible outside of the air conditioning unit to protect the eyes or skin of the occupants of the vehicle.

In some implementations, the UV sterilization module 202 can include a plurality of UV LED lights arranged within the vortex or plenum of the vehicle air conditioner (e.g., arranged in a corkscrew shape or other suitable shape). In some implementations, the UV sterilization module 202 can be activated when the air conditioner fan (or room air blower) is running. The UV sterilization module 202 can be used to reduce or help prevent microorganisms in the air in the vehicle or air being brought in from the outside such as bacteria or viruses, or other pollutants such as mold spores.

FIG. 3 is a diagram showing an example UV sterilization component in accordance with some implementations. In particular, FIG. 3 shows a UV sterilization module 300 that includes a UV light source 302 (e.g., one or more UV LEDs, or a UV tube) arranged behind a baffle 304 that helps reduce or prevent UV light from escaping the vehicle air conditioner. The UV light source is shown as a linear configuration of LED lights. Other light types and configurations can be used. Also, the baffle 304 is shown diagrammatically for illustration purposes and other shapes or configuration can be used.

In addition to use in a vehicle such as a recreational vehicle (or RV or camper), a boat, or another vehicle such as a car, bus or van, a UV sterilization module can be used to augment a water triple filter to help destroy microorganisms in the water. Also, a UV sterilization module as disclosed herein can be used in a commercial dishwashing unit and on or around a cutting board to help kill microorganisms on dishes and on the cutting board.

While some example implementations have been described in terms of a general embodiment with several specific example modifications, it is recognized that other modifications and variations of the embodiments described above are within the spirit and scope of the disclosed subject matter. Applicant intends to embrace any and all such modifications. variations and embodiments.

What is claimed is:

1. A recreational vehicle air conditioning system comprising:
an ultraviolet (UV) sterilization module having a UV light source disposed in the vortex of the recreational vehicle air conditioning system,
wherein the UV sterilization module is constructed such that the incoming air is exposed to UV light emanating from the UV light source at a level that inhibits growth of, or destroys, microorganisms in the air, and
wherein the UV light is not visible outside of the recreational vehicle air conditioning system so as to protect the eyes or skin of the occupants of the recreational vehicle from UV light exposure.

2. The recreational vehicle air conditioning system of claim 1, wherein the UV light source includes a plurality of UV LED lights arranged within the vortex or plenum of the recreational vehicle air conditioning system.

3. The recreational vehicle air conditioning system of claim 2, wherein the plurality of UV LED lights are arranged in a corkscrew shape.

4. The recreational vehicle air conditioning system of claim 1, wherein the UV sterilization module is activated when the recreational vehicle air conditioning system fan is running.

5. The recreational vehicle air conditioning system of claim 2, wherein the UV sterilization module is activated when the recreational vehicle air conditioning system fan is running.

6. The recreational vehicle air conditioning system of claim 1, further comprising a baffle, wherein the UV light source is arranged behind a baffle to reduce or prevent UV light from escaping the recreational vehicle air conditioning system.

7. The recreational vehicle air conditioning system of claim 1, wherein the UV light source includes one or more of a UV LED or a UV tube.
